(19) United States
(12) Patent Application Publication
(10) Pub. No.: US 2026/0355121 A1
(43) Pub. Date: Nov. 12, 2026

(54) LIPID NANOPARTICLES FOR ENHANCED PULMONARY DELIVERY OF NUCLEIC ACID-BASED THERAPIES

(71) Applicant: University of Maryland, College Park, College Park, MD (US)

(72) Inventors: Gregg Duncan, Columbia, MD (US); Sarah Samir Mahmoud Nasr, College Park, MD (US)

(21) Appl. No.: 19/671,888

(22) Filed: May 8, 2026

Related U.S. Application Data
(60) Provisional application No. 63/802,101, filed on May 8, 2025.

(51) Int. Cl. A61K 9/51; C12N 15/11; C12N 15/88

(52) U.S. Cl. CPC A61K 9/5123; A61K 9/5146; A61K 9/5169; C12N 15/111; C12N 15/88; C12N 2310/20

(57) ABSTRACT

The current disclosure provides novel lipid nanoparticles comprising lung surfactants (Surf-LNPs) wherein said Surf-LNPs are useful for providing transfer of genetic material, i.e., nucleic acids, into cells and/or tissues of the lung or potentially other organs/tissues. Surf-LNPs comprise one or more biocompatible lipid components, one or more lung surfactants, and one or more nucleic acids encoding therapeutic agents, prophylactic agents or a combination thereof. The present disclosure further provides pharmaceutical compositions comprising said Surf-LNPs and their use in the treatment of lung disease. Said Surf-LNPs may also serve as vaccine compositions through the incorporation of nucleic acids therein which encode an immunogen or antigen of interest.

### PRIORITY

This application claims benefit and priority to U.S. Provisional Application No. 63/802,101, filed May 8, 2025, which is incorporated herein by reference in their entirety.

### GOVERNMENT SUPPORT STATEMENT

This invention was made with government support under HL160540 awarded by the National Institutes of Health. The government has certain rights in the invention.

### TECHNICAL FIELD

The current disclosure provides novel surfactant-enhanced lipid nanoparticles comprising lung surfactants (Surf-LNPs) wherein said Surf-LNPs are useful for providing transfer of genetic material, i.e., nucleic acids, into cells and/or tissues of the lung or potentially other organs/tissues. Surf-LNPs comprise one or more biocompatible lipid components, one or more lung surfactant components, and one or more nucleic acids encoding therapeutic agents, prophylactic agents, a combination thereof or silencing RNA (siRNA). The present disclosure further provides pharmaceutical compositions comprising said Surf-LNPs and their use in the treatment of lung disease. Said Surf-LNPs may also serve as vaccine compositions through the incorporation of nucleic acids therein which encode an immunogen or antigen of interest.

### BACKGROUND OF THE INVENTION

Compositions and methods useful in the management and treatment of a large number of diseases and disorders, such as pulmonary diseases, are needed. This includes pulmonary cancers and refractory obstructive respiratory diseases like asthma and chronic obstructive pulmonary disorders as well as diseases and disorders resulting from protein, lipoprotein (such as surfactant lipoproteins SP-A, SP-B, SP-C or SP-D) and/or enzyme deficiencies. Individuals suffering from such protein or enzyme deficiencies may have underlying genetic defects that lead to alterations in expression of a protein or enzyme, including, for example, the non-synthesis of the protein, the reduced synthesis of the protein, or synthesis of a protein lacking or having diminished biological activity. For example, a majority of pulmonary congenital diseases, such as cystic fibrosis (CF), respiratory distress syndrome (RDS) and α-1 antitrypsin deficiency, are caused by genetic mutation. Indeed, CF is a genetic disorder affecting more than 70,000 people worldwide, due to mutations in the cystic fibrosis transmembrane conductance regulator (CFTR) gene. CFTR dysfunction leads to airway plugging, chronic infections, and progressive declines in pulmonary function.

Pulmonary delivery of therapeutic nucleic acids (transgenes) by inhalation demands delicate formulation engineering and manufacturing. Pulmonary delivery of conventional LNPs has not proven as successful with inflammatory effects noted in pre-clinical studies. These challenges suggest alternatives in LNP design are necessary to recognize their potential as inhaled gene therapy. Pulmonary disease remains an enormous health care burden and an extremely large unmet medical need and compositions and, accordingly, methods for inhaled gene delivery in the lung are greatly needed.

### SUMMARY

The current disclosure provides novel surfactant-enhanced lipid nanoparticles (LNPs) comprising lung surfactants (Surf-LNPs) wherein said Surf-LNPs are useful for providing transfer of genetic material, i.e., nucleic acids, into cells and/or tissues of the lung. Said Surf-LNPs comprise one or more biocompatible lipid components as well as one or more lung surfactant components. The present disclosure further provides that the Surf-LNP is associated with a nucleic acid that encodes one or more therapeutic agents, prophylactic agents or a combination thereof.

In one aspect, the nucleic acid can be custom-synthesized to contain properties that are tailored to fit the desired use. Said nucleic acids (transgenes) may be engineered to express proteins or polypeptides that function to treat or prevent a disease, such as for example, a pulmonary disease. In an embodiment, the expressed protein or polypeptide may be one whose expression is found to be defective, deficient, or missing in a subject due to a genetic defect. The transgenes may be additional copies of genes already present in the cell and are transferred to increase the amount of expression product of the transgene. The transgene may express the required gene products in the body, for therapeutic purposes. The transgene can alter, correct, or replace an endogenous nucleic acid sequence.

In a specific embodiment, the transgene comprises one or more therapeutic nucleic acids for treatment or prevention of diseases such as pulmonary diseases. The nucleic acid carried by the Surf-LNP can be a DNA, RNA, or combinations thereof. Such nucleic acids include oligonucleotides, polynucleotides and mixtures thereof. In embodiments, the nucleic acid molecule is a DNA plasmid, cDNA, mRNA, siRNA, CRNA, saRNA, PNA or related species, microRNA, an antisense RNA molecule or a single guide RNA (sgRNA) nucleic acid to be used in conjunction with the CRISPR/Cas9 system.

In an embodiment, the transgene comprises a chemically modified nucleic acid. For example, methods for increasing stability of nucleic acid half-life and resistance to enzymatic cleavage are known in the art and can include one or more modifications or substitutions to the nucleobases, sugars, or linkages of the polynucleotide.

In a specific embodiment, the Surf-LNPs may comprise the components of a CRISPR-Cas9 system designed for correction of a genetic defect that results in pulmonary disease. For example, a CRISPR-Cas9 system may be designed to correct the genetic defect in the cystic fibrosis gene responsible for the symptoms of the disease. In such an instance, the transgene can encode a normal cystic fibrosis transmembrane conductance regulator (CFTR). In other preferred embodiments, the transgene is used to correct defects in the CFTR genes. Said correction can be accomplished through the use of a CRISPR/Cas9 system or other gene editing systems, in vivo or ex vivo where the sgRNA is designed to target the defective gene(s) for correction of the genetic defect.

In one aspect, the present disclosure further provides pharmaceutical compositions comprising Surf-LNPs and a pharmaceutically acceptable carrier. The carrier typically includes a diluent, a buffer system, an excipient, a stabilizer, a preservative, and the like. In a specific embodiment, the Surf-LNP pharmaceutical composition is formulated for inhalation or intranasal administration. Such inhalation or intranasal administration may be accomplished using, for example, a nebulizer or inhaler.

In one aspect, a method of treating a subject having a pulmonary disease or disorder is provided that includes administering a Surf-LNP composition, as described herein, to a subject in need thereof. In a non-limiting embodiment, the Surf-LNP composition is able to reduce the severity of symptoms or eliminate the symptoms of pulmonary disease. Thus, for methods of treatment provided herein, the Surf-LNP may be administered prophylactically to a subject, to prevent the pulmonary disease, or may be administered to subjects after the development of the pulmonary disease.

Pulmonary diseases to be treated include, for example, airway diseases such as asthma, COPD (including emphysema and chronic bronchitis), bronchiolitis, and bronchiectasis; pulmonary tissue diseases such as pulmonary fibrosis, sarcoidosis, and interstitial pulmonary disease (ILD); pulmonary circulation diseases such as pulmonary hypertension; pulmonary infections such as pneumonia, influenza, and tuberculosis; and pulmonary cancers including mesothelioma. In addition, the one or more Surf-LNPs may be used for treatment of pulmonary diseases that result from a genetic defect, said diseases including, for example, cystic fibrosis, alpha-1 antitrypsin deficiency, Respiratory Distress Syndrome (RDS) and primary ciliary dyskinesia (PCD).

In another aspect, the present disclosure provides Surf-LNPs as immunogenic compositions comprising an effective amount of nucleic acid encoding for an antigen or a hapten of interest. A vaccine comprising the immunogenic composition is also provided.

Generally, the formulations are administered to the pulmonary tract. Aerosolized pharmaceutical formulations can be delivered to the lungs, preferably using a device, such as nebulizer, ventilator or pressurized metered dose inhaler (pMDI). Liquid formulations can also be administered to the respiratory tract by other suitable methods such as intranasal instillation, intranasal sprays intratracheal instillation, and intratracheal injection.

The present disclosure further provides a method for making the Surf-LNPs disclosed herein. Such methods, well known those of skill in the art include, for example, nanoprecipitation, ethanol injection, spontaneous electrostatic assembly, coacervation, ultrasonication, homogenization, film hydration, microfluidic mixing, Impingement jet mixing, T-junction mixing, supercritical fluid methods, chemical reduction, coprecipitation, seeding, microemulsion, inverse microemulsion, hydrothermal method, and sonic deposition. In one aspect, the method for preparation of Surf-LNPs as has been defined above, comprises the steps of: (i) dissolving lipids in ethanol, (ii) dissolving the nucleic acid in an acidic aqueous buffer (iii) mixing (i) & (ii) to generate Surf-LNPs, and (iii) dialyzing the prepared Surf-LNPs or adopting other methods for solvent exchange. In a non-limiting embodiment, the aqueous/organic phase ratio ranges from 1:1 to 10:1 and the N:P ratio ranges from 2:1 to 20:1.

The present disclosure provides kits that include the Surf-LNP compositions, as described herein. Such kits may be used for treatments or as vaccines as described herein. In one specific aspect the kit further includes instructions for the treatment and/or prophylaxis of pulmonary disease. The Surf-LNP compositions may, if desired, be presented in a pack or dispenser device which may contain one or more unit dosage forms containing compositions. In a specific embodiment, the dispenser may be one to be used for inhalation administration.

### BRIEF DESCRIPTION OF THE DRAWINGS

Various embodiments of methods are described herein with reference to the drawings wherein:

FIG. 1A-E. (FIG. 1A) Diagrammatic illustration of LNP vs Surf-LNP composition. PC: phosphatidyl choline, PL: phospholipids, PG: phosphatidyl glycerol. Physicochemical properties of Surf-LNPs vs LNPs characterized using Nanoparticle Tracking Analysis (NTA) to determine (FIG. 1B) particle diameter, (FIG. 1C) zeta potential, and (FIG. 1D) particle concentration (particles/mL). (FIG. 1E) mEGFP encapsulation efficiency in LNP vs Surf-LNPs assessed using Ribogreen assay.

FIG. 2A-C. (FIG. 2A) Schematic of LNP diffusion through the mucus barrier. Multiple Particle Tracking of LNPs vs Surf-LNPs in mucus harvested from BCi-NS1. (FIG. 2B) Representative trajectories and (FIG. 2C) histogram of mean squared displacement (MSD) at time scale of 1 s for LNP and Surf-LNP diffusion in mucus.

FIG. 3A-F. In vitro assessment of LNP and Surf-LNP delivery of mRNA. (FIG. 3A) Transfection efficiency (% mEGFP cells) and (FIG. 3B) geometric mean fluorescent intensity (gMFI) in A549 lung epithelial cells following treatment with Monomeric Enhanced Green Fluorescent Protein (mEGFP) mRNA loaded LNP and Surf-LNP. (FIG. 3C) Cytotoxicity of LNPs vs Surf-LNPs in A549 lung epithelial cells. (FIG. 3D) Transfection efficiency (% mEGFP+ cells) and (FIG. 3E) geometric mean fluorescent intensity (gMFI) in BCi-NS1.1 lung basal cells following treatment with EGFP-mRNA loaded LNP and Surf-LNP. (FIG. 3F) Cytotoxicity of LNPs vs Surf-LNPs in BCi-NS1.1. Data is displayed as Mean±SD, (N=3(A, B) or 1(C), n=3), ns=non-significant, *p<0.05, **p<0.01, ***p<0.001, and ****p<0.0001.

FIG. 4A-C. Colocalization analysis of LNP/Surf-LNP with endosomal compartments. (FIG. 4A) CLSM images demonstrating co-localization of DiD labelled LNP/Surf-LNP with Lysotracker Yellow HCL-123 staining endosomal compartments at 2 h and 6 h following LNP/Surf-LNP treatment in BCi-NS1 (Scale bar=50 μM) (FIG. 4B) Quantitative analysis of colocalization using Pearson's correlation coefficient r=0 indicates no co-localization, r=1 indicates complete endosomal co-localization). (FIG. 4C) Apparent pKa of LNP vs Surf LNP. ARF; Average Relative Fluorescence.

FIG. 5A-D. In vivo assessment of LNP and Surf-LNP delivery of mRNA. (FIG. 5A) CLSM images of the trachea of Balb-c mice after 6 h or 24 h of treatment with mCherry mRNA loaded LNP and Surf-LNP, respectively (scale bar=50 μm). (FIG. 5B) Transfection efficiency represented as % mCherry positive cells normalized to the total number of cells (DAPI) in CLSM. (FIG. 5C) mCherry fluorescence in lung homogenates of untreated, LNP, and Surf-LNP treated groups. (FIG. 5D) in vivo transfection efficiency of epithelial (EPCAM+) vs immune (CD45+) cells by LNPs vs Surf-LNPs. RFU; Relative Fluorescence Units

FIG. 6A-B. (FIG. 6A) Comparative compositions of physiological human lung surfactant vs commercially available porcine lung surfactant cocktail Poractant-α. (FIG. 6B) Chemical structures of DSPC (helper lipid in commercially available LNPs) and DPPC (main phospholipid component in Poractant-α).

FIG. 7. Particle stability demonstrated as a function of changes in particle count over time following 4 h incubation in simulated lung fluid (Gamble's solution).

FIG. 8. In vitro assessment of LNPs vs Surf-LNPs in A549 cells. Confocal images demonstrating GFP expression in untreated, LNP-treated, and Surf-LNP-treated A549 (scale bar=50 μm).

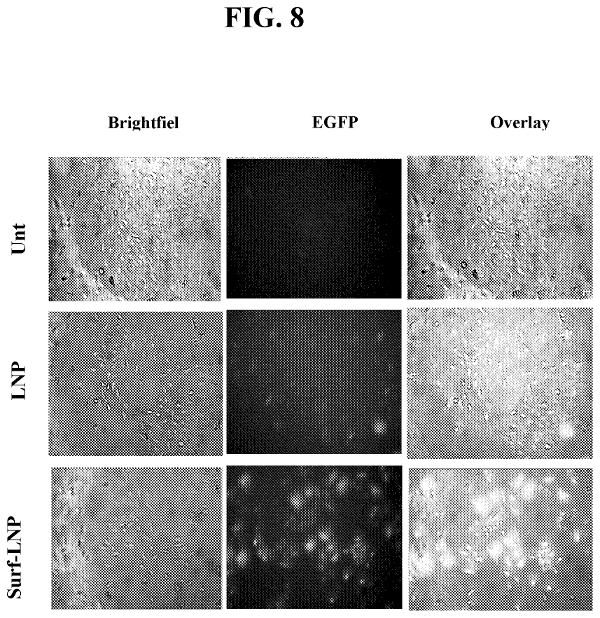

FIG. 9. Representative confocal images of lung parenchyma of Balb-c mice after 24 h of treatment, respectively (scale bar=50 μm)

FIG. 10. Cytokine levels in lung tissue homogenates from LNP and Surf-LNP treated mice measured by ELISA.

### DETAILED DESCRIPTION

The practice of the present disclosure will employ, unless otherwise indicated, conventional techniques of nanotechnology, nano-engineering, molecular biology (including recombinant techniques), microbiology, cell biology, biochemistry, immunology, and pharmacology, which are within the skill of the art. Unless defined otherwise, all technical and scientific terms used herein have the same meaning as is commonly understood by one of ordinary skill in the art to which this disclosure belongs.

The current disclosure provides novel lipid nanoparticles (LNPs) comprising lung surfactants (Surf-LNPs) wherein said Surf-LNPs are useful for providing transfer of genetic material, i.e., nucleic acids, into cells and/or tissues of the lung. In one aspect, the Surf-LNP comprises one or more biocompatible lipid components as well as one or more lung surfactants' components. In another aspect, the Surf-LNP comprises a nucleic acid of interest. The present disclosure provides novel nanoparticles, and methods of using and making thereof.

The term “nanoparticle” as used herein refers to nanostructure, particles, vesicles or fragments thereof having at least one dimension (e.g., height, length, width, or diameter) of between about 500 nm and about 1 nm. In certain embodiments, the nanoparticles provided herein are biocompatible and/or biodegradable.

In a specific embodiment the Surf-LNP may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-di-(9Z-octadecenoyl)-sn-glycero-3-phosphoethanolamine (DOPE), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-di-O-octadecenyl-3-trimethylammonium propane (DOTMA), 1,2-dioleyloxy-3-dimethylaminopropane (DODMA), unsaturated, multivalent-tail, branched-tail, polymeric, biodegradable ionizable lipids or lipidoids including but not limited to (6Z,9Z,28Z,31Z)-heptatriaconta-6,9,28,31-tetraen-19-yl 4-(dimethylamino)butanoate (Dlin-MC3-DMA), 8-[(2-hydroxyethyl) [6-oxo-6-(undecyloxy)hexyl]amino]-octanoic acid, 1-octylnonyl ester (SM-102), 2-hexyl-decanoic acid, 1,1′-[[(4-hydroxybutyl)imino]di-6,1-hexanediyl] ester (ALC-0315), bis(2-butyloctyl) 10-(N-(3-(dimethylamino)propyl)nonanamido)nonadecanedioate (A9), heptadecan-9-yl (Z)—N-(((4-(dimethylamino)butyl)thio)carbonyl)-N-(2-(non-2-en-1-yloxy)-2-oxoethyl)glycinate (ATX-0114/Lipid 2,2 (8,8) 4C CH3), 5-(dimethylamino)-pentanoic acid, (6Z)-1,2-di-(4Z)-4-decen-1-yl-6-dodecen-1-yl ester (Lipid 10/CL1), 9Z,12Z-octadecadienoic acid, 3-[4,4-bis(octyloxy)-1-oxobutoxy]-2-[[[[3-(diethylamino)propoxy]carbonyl]oxy]methyl]propyl ester (LP01/CIN-16645), 3,6-bis[4-[bis[(9Z,12Z)-2-hydroxy-9,12-octadecadien-1-yl]amino]butyl]-2,5-piperazinedione (OF-02), 9-[4-(dimethylamino)-1-oxobutoxy]-heptadecanedioic acid, 1,17-di-3-decyn-1-yl ester (A6), Ethyl 1-[3-(2-ethyl-1-piperidinyl) propyl]-5,5-di-(8Z)-8-heptadecen-1-yl-2,5-dihydro-1H-imidazole-2-carboxylate (A18-Iso5-2DC18), N,N-ditetradecylamine-polylysine (40) (N-TETAMINE-pLys40), N1-[2-((1S)-1-[(3-aminopropyl)amino]-4-[di(3-amino-propyl)amino]butylcarboxamido)ethyl]-3,4-di[oleyloxy]-benzamide (MVL5),2,3-dioleyloxy-N-[2-(sperminecarboxamido)ethyl]-N,N-dimethyl-1-propanaminium HCl salt (DOSPA), 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200),N1,N16-didodecyl-4,7,13-tris[3-(dodecylamino)-3-oxopropyl]-4,7,10,13-tetraazahexadecanediamide (98N12-5/ND98),3,6-bis[4-[bis(2-hydroxydodecyl)amino]butyl]-2,5-piperazinedione (cKK-E12), 2-(dioctylamino)ethyl nonyl hydrogen phosphate (9A1P9), poly(ethyleneamine) with C15 lipid (7C1),3,3′,3″,3′″-(ethane-1,2-diylbis(azanetriyl))tetrakis(N-(2-(bis(2-hydroxytetradecyl)amino)ethyl) propanamide) (GO-C14 or analogs), 9-[4-(dimethylamino)-1-oxobutoxy]-heptadecanedioic acid, 1,17-di-(2Z)-2-nonen-1-yl ester (L-319), Tri-N-tridecyl 3-(ethyl(methyl)amino)propanoate (304O₁₃), 9Z,12Z-octadecadienoic acid, 1,1′,1″,1″′-[(3,6-dioxo-2,5-piperazinediyl)bis(4,1-butanediylnitrilodi-2,1-ethanediyl)] ester (OF-Deg-Lin), tetrakis(2-(octyldisulfaneyl)ethyl) 3,3′,3″,3′-(((methylazanediyl)bis(propane-3,1-diyl))bis(azanetriyl))tetrapropionate (306O12B), tetrakis(8-methylnonyl) 3,3′,3″,3′-(((methylazanediyl)bis(propane-3,1-diyl))bis(azanetriyl))tetrapropionate (3060i10), 9,9′,9″,9′″,9″″,9″″′-[1,3,5-benzenetriyltris(carbonylimino-3,1-propanediylnitrilo)]hexakis-nonanoic acid, 1,1′,1″,1″′,1″″,1″″′-hexakis (1-ethylhexyl) ester (FTT5).

In one embodiment, the Surf-LNP has DSPC replaced with lung surfactants, such as Poractant-α, as described in detail below.

Cholesterol may also be included as well as relevant derivatives or alternatives, including but not limited to phytosterols (e.g. C-24 alkyl phytosterols), Hchol (3β[L-histidinamide-carbamoyl]cholesterol), 7α-hydroxycholesterol, Cholic acid, Corosolic acid derivative (CAβ), Bile acids.

Anionic lipids may also be incorporated and examples include but are not limited to 1,2-dimyristoyl-sn-glycero-3-phosphate (sodium salt) (14PA) and sn-(3-oleoyl-2-hydroxy)-glycerol-1-phospho-sn-3′-(10,20-dioleoyl)-glycerol (ammonium salt) (18BMP), 1,2-dioleoyl-sn-glycero-3-phosphate (18PA).

Pegylated lipids including but not limited to 1,2-dipalmitoyl-sn-glycero-3-phosphoethanolamine-N [methoxy (polyethylene glycol)-2000] (DPPE-PEG2000), 1,2-distearoyl-sn-glycero-3-phosphoethanolamine-N-[methoxy (polyethylene glycol)-2000] (DSPE-PEG2000), 1,2-dimyristoyl-rac-glycero-3-methoxypolyethylene glycol-2000 (DMG-PEG 2000), 1,2-distearoyl-sn-glycero-3-phosphoethanolamine-N-hydrazone-[methoxy(polyethylene glycol)] (DSPE-Hydrazone-PEG) may also be included. Less immunogenic PEG alternatives may also be employed, examples of which include but are not limited to poly(vinyl pyrrolidone), polysarcosine, reversible addition-fragmentation chain transfer (RAFT) lipopolymers, polysaccharides, poly (amino acids), XTEN polymers, Poly (thioglycidyl glycerol).

In a first aspect, the present disclosure provides Surf-LNPs comprising one or more lipid components, as well as one or more lung surfactants. In an embodiment, the lung surfactant may comprise one or more of the following components: phosphatidylcholine (PC), phosphatidyl glycerol, cholesterol, cholesterol derivatives, or cholesterol alternatives, and amphiphilic surfactant proteins such as SP-A, SP-B, SP-C and SP-D. In one embodiment the lung surfactant is Poractant-α (as referred to herein as Curosurf). In additional embodiments, several other animal-derived (bovine or porcine) and synthetic surfactants can be included in the Surf-LNPs disclosed herein. Examples of such lung surfactants, include but are not limited to, Beractant (Survanta), Calfactant (Infasurf), Bovine Lipid Extract Surfactant (BLES), Alveofact, Surfacen, Colfosceril palmitate, Lucinactant, Pumactant, Sinaplutide, Venticute, Varasurf or Bovacant. Such lung surfactants are commercially available.

In one embodiment, the Surf-LNP comprises one or more of the following components (i) an ionizable lipid; (ii) cholesterol, cholesterol derivative, or cholesterol alternative; (iii) a helper lipid; (iv) a PEGylated lipid: (v) a lung surfactant cocktail or component(s) and (vi) one or more sorting lipids.

Ionizable lipids or lipidoids are a component of the disclosed Surf-LNPs, due to their ability to facilitate the delivery of nucleic acids into cells. These LNPs are designed to be positively charged at acidic pH, enabling them to bind to negatively charged mRNA and other nucleic acids, forming a stable complex. This complex is then taken up by cells, where the acidic endosomal environment causes the ionizable lipid to become protonated, facilitating the endosomal escape of the LNP in part or whole and the release of the mRNA cargo into the cytoplasm. Such ionizable lipids or lipidoids include but are not limited to, ALC-0315, C12-200, DLin-MC3-DMA, and cKK-E12, SM-10, A9, ATX-0114 (Lipid 2,2(8,8) 4C CH3), LP01 (CIN-16645), Lipid 10 (CL1), OF-02, A6, A18-Iso5-2DC18, MVL5, DOSPA, 98N12-5 (ND98), 9A1P9, GO-C14, L-319,3040₁₃, OF-Deg-Lin, 306012B, 306Oi10, FTT5 or analogs.

Helper Lipids are a class of lipid molecules that increase particle stability and fluidity of lipid nanoparticles (LNP). Examples include but are not limited to Phosphatidylcholines (e.g. DSPC (1,2-distearoyl-sn-glycero-3-phosphocholine) and HSPC (hydrogenated soybean phosphatidylcholine), Phosphatidylethanolamines (e.g.DOPE (1,2-dioleoyl-sn-glycero-3-phosphoethanolamine), DOPC (1,2-dioleoyl-sn-glycero-3-phosphocholine), Phosphatidylserines (or other anionic helper lipids), DOTAP (1,2-dioleoyl-3-trimethylammonium propane) (or other cationic helper lipids).

In one non-limiting embodiment, LNPs are formed with ionizable lipids such as dilinoleyl-methyl-4-dimethylaminobutyrate MC3 (10-50% molar ratio), cholesterol (5-40% molar ratio), helper lipids (DSPC; 0-30%), and PEGylated lipids (2 kDa PEG; 0-70% molar ratio). Surf-LNPs are similarly prepared with 0-100% substitution of DSPC or any of the LNPs other lipid components with a lung surfactant cocktail or component.

The present disclosure further provides a method for making the nanoparticles disclosed herein. Such methods, well known to those of skill in the art include, for example, nanoprecipitation, ethanol injection, spontaneous electrostatic assembly, coacervation, ultrasonication, homogenization, film hydration, microfluidic mixing, Impingement jet mixing, T-junction mixing, supercritical fluid methods, conventional mixing, dissolving, emulsifying, encapsulating, entrapping or lyophilizing processes, chemical reduction, coprecipitation, seeding, microemulsion, inverse microemulsion, hydrothermal method, and sonic deposition. Microfluidic mixing methods considered as state of the art are most likely to be adopted given their reproducibility, scalability, and their provision of laminar flow conditions to minimize shear stress and LNP disruption. In such methods, an organic phase containing lipid components of the formulation is mixed with an aqueous phase containing the hydrophilic components (including the nucleic acid cargo) of the formulation, on microfluidic chips/mixers of suitable geometry. Mixing parameters including but not limited to organic to aqueous phase ratio (v/v), mixing times, flow rate ratios, solutions' concentrations, N/P (Nitrogen to Phosphate) ratios are adjusted as per the individual requirements of the formulation.

The present disclosure further provides that the Surf-LNP comprises one or more nucleic acid molecules. Said nucleic acids may encode therapeutic agents, prophylactic agents, or a combination thereof. For example, the nucleic acids can be custom-synthesized to contain properties that are tailored to fit a desired use. Said nucleic acids (transgenes) may be engineered to express proteins or polypeptides that function to treat or prevent a targeted disease or disorder. In an embodiment, the disease or disorder is a pulmonary disorder. In one aspect, the expressed protein or polypeptide may be one whose expression is found to be deficient or missing in a subject due to a genetic defect. The transgenes may be additional copies of genes already present in the cell and are transferred to increase the amount of expression product of the transgene. The transgene may express the required gene products in the body, for therapeutic purposes. The nucleic acid can alter, correct, or replace an endogenous nucleic acid sequence.

In a specific embodiment, the therapeutic nucleic acids may be used for treating or preventing a disease or disorder in a subject in need. In such an instance, the Surf-LNP is designed to deliver a therapeutic nucleic acid of interest to lung cells and tissues of a subject wherein the nucleic acid is expressed in the lung to provide a therapeutic agent to said subject. Treatment and prevention is provided of any physiological or pathological conditions, including, but not limited to, an infectious disease, a parasitic disease, a neoplasm, a disorder involving the immune mechanism, endocrine, nutritional and metabolic diseases, inflammatory disease, diseases of the nervous, circulatory, respiratory, digestive, musculoskeletal or circulatory system, diseases of the skin and subcutaneous tissue, to name a few. In a specific embodiment, the Surf-LNP is designed to deliver a therapeutic nucleic acid of interest to lung cells and tissues of a subject for treatment of pulmonary lung disease.

The nucleic acid carried by the Surf-LNP can be DNA, RNA, peptide nucleic acids (PNAs), locked nucleic acids (LNAs), or combination thereof. Such nucleic acids include oligonucleotides, polynucleotides and mixtures thereof. In embodiments, the nucleic acid molecule is a DNA plasmid, cDNA, mRNA, self-amplifying mRNA, CRNA, saRNA, PNA or related species, siRNA, microRNA, an antisense RNA molecule or an antisense oligonucleotide (ASO).

In an embodiment, the transgene comprises a chemically modified nucleic acid. For example, methods for increasing stability of nucleic acid half-life and resistance to enzymatic cleavage are known in the art and can include one or more modifications or substitutions to the nucleobases, sugars, or linkages of the polynucleotide. For example, LNP modifications involve a locking of the sugar ring in a conformation that stabilizes the duplex and increases its thermal stability: replacing a non-bridging oxygen atom in the phosphate backbone with sulfur can enhance nuclease resistance, preventing degradation; modifications that add a methylene group to the sugar backbone can further increase nuclease resistance, particularly against both 3′ and 5′ exonucleases; introducing O-methoxyethyl groups can improve thermal stability, nuclease resistance, and binding affinity to the RNA interference (RNAi) machinery; replacing the hydrogen at the C-5 position of pyrimidines can significantly increase duplex stability; and altering the phosphodiester linkage (the bond between nucleotides) can enhance nuclease resistance and overall stability.

In one embodiment, a vector, preferably an expression vector, comprising one or more nucleic acids encoding a protein of interest to be transferred to lung cells or tissue, or fragments thereof, is provided. Methods are well known to one of skill in the art and can be used to construct expression vectors containing the protein coding sequence with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). Typically, the vectors are derived from virus, plasmid, prokaryotic or eukaryotic chromosomal elements, or some combination thereof, and may optionally include at least one origin of replication, at least one site for insertion of heterologous nucleic acid, and at least one selectable marker. Preferably, the vector is a viral vector, such as a lenti- or baculo- or preferably adeno-viral/adeno-associated viral vectors.

In one embodiment, the present disclosure relates to Surf-LNP compositions that comprise nucleic acid molecules designed to target a specific mRNA and inhibit, silence or attenuate the expression of that RNA and methods for preparing them. In such instances, the nucleic acid molecules contain a region of nucleotide sequence that can direct the destruction and/or translational inhibition of the targeted RNA transcript. Methods for design and expression of such nucleic acids, e.g., antisense, miRNA, siRNA and shRNA, are well known to those of skill in the art. The target RNA for inhibition, silencing or attenuation may be one in which the expression of is associated with a pulmonary disease or disorder. Accordingly, such compositions may be used in methods for prevention or treatment of pulmonary disease or disorders.

The terms “inhibit,” “silencing,” and “attenuating” can refer to a measurable, e.g., quantifiable amount, reduction in expression of a target mRNA (or the corresponding polypeptide or protein) as compared with the expression of the target mRNA (or the corresponding polypeptide or protein) in the absence of an interfering RNA molecule. The reduction in expression of the target mRNA (or the corresponding polypeptide or protein) is commonly referred to as “knock-down” and is reported relative to levels present following administration or expression of a non-targeting control RNA.

The term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways, wherein the compounds interfere with gene expression.

The term “shRNA” refers to any small hairpin RNA or short hairpin RNA. Although it is not necessary to understand the mechanism of action, it is believed that any sequence of RNA that makes a tight hairpin turn can be used to silence gene expression via RNA interference. Typically, shRNA uses a vector introduced into a cell genome and is constitutively expressed by a compatible promoter. The shRNA hairpin structure may also be cleaved into siRNA, which may then become bound to the RNA-induced silencing complex (RISC). This complex binds to and cleaves mRNAs which match the siRNA that is bound to it.

The term “microRNA” or “miRNA”, refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

In another embodiment, the Surf-LNPs may be designed to be associated with the components of a CRISPR-CAS9 or other gene editing systems designed for correction of a genetic defect that results in pulmonary disease. The main components of a CRISPR-Cas system are a guide RNA (gRNA) or single-guide RNA (sgRNA) and a CRISPR-associated (Cas) nuclease. The gRNA directs the Cas nuclease to a specific DNA sequence, and the nuclease then cleaves the DNA at that site, allowing for gene editing. In engineered systems, the gRNA is often combined with a tracrRNA sequence, which helps the Cas nuclease bind and facilitates the processing of mature crRNAs. This combined RNA is referred to as an sgRNA.

In one aspect, the invention provides methods for using one or more elements of a CRISPR-Cas or other gene editing systems for expression of a wild-type protein. Included are Surf-LNPs comprising vectors and vector systems some of which encode one or more components of a CRISPR complex, as well as methods for the design and use of such vectors.

The CRISPR complex of the invention provides an effective means for modifying a target polynucleotide. The CRISPR complex has a wide variety of utilities including modifying (e.g., deleting, inserting, translocating, inactivating, activating) a target polynucleotide in lung cells and tissue. As such the CRISPR complex of the invention has a broad spectrum of applications in, e.g., gene or genome editing and gene therapy.

In one embodiment, the Surf-LNP associated nucleic acid encodes a normal cystic fibrosis transmembrane conductance regulator (CFTR). In other preferred embodiments, the nucleic acid is used to correct defects in the CFTR genes. Said correction may be accomplished using a CRISPR/CAS9 system where the sg is designed to target the CTFR gene for correction of the genetic defect. In another embodiment the Surf-LNP contains a nucleic acid encoding the CAS9 enzyme, or variants thereof.

Sequence analysis of the CFTR gene of CF chromosomes has revealed a variety of disease-causing mutations. For example, the most prevalent mutation is a deletion of phenylalanine at position 508 of the CFTR amino acid sequence and is commonly referred to as ΔF508-CFTR. This mutation occurs in approximately 70 percent of cases of cystic fibrosis and is associated with a severe disease. Other target mutations include antisense mutations (Class I) or CFTR trafficking defects (Class V, VI) which show limited response to state-of-the-art small molecule drug treatments (e.g.Ivacaftor, Lumacaftor, Tezacaftor). All the aforementioned mutation classes as well as others not mentioned specifically herein, can be adressed by Surf-LNP delivered mRNA, self-amplifying mRNA, and gene editing systems in a mutation agnostic manner.

In one aspect, CFTR-encoding mRNA, may be delivered to lung epithelial cells to prompt them to express the healthy CFTR form in therapeutically efficient amounts. There are at least five CFTR modulators for use in treating subjects with certain CFTR mutations: Alyftrek (vanzacaftor/tezacaftor/deutivacaftor) Trikafta (elexacaftor/the zacaftor/ivacaftor) Symdeko (tezacaftor/ivacaftor) Orkambi (lumacaftor/ivacaftor) or Kalydeco (ivacaftor) The effects of CFTR modulators only last for as long as the medication is present in the treated subject's body. Accordingly, the medication, e.g. CFTR modulators, have to be taken repeatedly or as the care team prescribes. In one aspect, the nucleic acid is a self-amplifying nucleic acid. The current disclosure improves on the existing treatments as the mRNA-based therapies intended for delivery using the Surf-LNP act in a mutation-type independent manner. Regardless of the mutation class, CFTR-encoding mRNA, if delivered to lung epithelial cells successively, can direct said cells to express the healthy CFTR form in functional amounts.

In one aspect, the present disclosure provides Surf-LNP pharmaceutical compositions that contain a pharmaceutically acceptable carrier. Pharmaceutical compositions comprise a therapeutically effective amount of one or more Surf-LNPs dissolved or dispersed in a pharmaceutically acceptable carrier. The preparation of a pharmaceutical composition that contains Surf-LNPs and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The disclosed pharmaceutical compositions are formulated to be administered to any mucosal surface in a patient to treat or lessen one or more symptoms associated with a pulmonary disease or disorder. Generally, the formulations are administered to the pulmonary tract, however, for other applications such as vaccination, or protein replacement therapies other routes of administration such as, for example, IV, IM, and intra-tumoral maybe considered. In a specific embodiment, the compositions are formulated for inhalation administration. Compositions for inhalation administration can be prepared so that, for example, the Surf-LNPs can be administered directly to the pulmonary mucosa. In a specific embodiment, the pharmaceutical compositions are formulated for intranasal administration. Intranasal administration, if used, is generally characterized by inhalation. Compositions for nasal administration can be prepared so that, for example, the Surf-LNPs can be administered directly to the mucosa (e.g., nasal and/or pulmonary mucosa). Said compositions may be formulated for administering as a nasal spray, medicated nasal swab, medicated wipe, nasal drops, or aerosol to the subject's nasal passages or nasal vestibule. In some embodiments, the compositions of present invention can be delivered using a nasal spray device, which can allow (self) administration with little or no prior training to deliver a desired dose. In some embodiments, nasal drops may be prepacked in pouches or ampoules that may be opened immediately prior to use and squeezed or squirted into the nasal passages.

Optionally, such inhalation compositions may further advantageously comprise a mucoadhesive, such as cellulose derivatives, polyacrylates, starch, chitosan, glycosaminoglycans, hyaluronic acid, and any combination thereof.

The compositions provided may be produced in an arbitrary unit dose. A unit dose refers to the amount, e.g., therapeutically effective amount, of the active ingredient and the pharmaceutically acceptable carrier contained in each product packaged for use in one or more administrations to a subject, such as a human, and an appropriate amount of such active ingredient and carrier is an amount that may function as a therapeutic for pulmonary disease when administration is performed one or more times, and such an amount may be determined non-clinically or clinically as understood by those skilled in the art.

The present disclosure provides methods for treatment of pulmonary diseases and disorders. Pulmonary diseases to be treated include, for example, airway diseases such as asthma, COPD (including emphysema and chronic bronchitis), bronchiolitis, and bronchiectasis; pulmonary tissue diseases such as pulmonary fibrosis, sarcoidosis, and interstitial pulmonary disease (ILD); pulmonary circulation diseases such as pulmonary hypertension; pulmonary infections such as pneumonia, influenza, and tuberculosis; and pulmonary cancers including mesothelioma. Treatments are provided for pulmonary diseases that result from a genetic defect, said diseases including, for example, cystic fibrosis, respiratory distress syndrome (RDS), alpha-1 antitrypsin deficiency, and primary ciliary dyskinesia (PCD).

A method of treating or preventing pulmonary disease in a subject is provided that includes administering the disclosed Surf-LNP compositions, as described herein, to a subject in need thereof. The terms “patient” and “subject” are used interchangeably and include human and non-human animal subjects. The terms “treat/treating/treatment” and “prevent/preventing/prevention” as used herein refers to eliciting the desired biological response, i.e., a therapeutic and prophylactic effect, respectively. In accordance with the present disclosure, the therapeutic effect includes one or more of a decrease/reduction in the severity of the disease (e.g., a reduction or inhibition of infection), a decrease/reduction in symptoms and disease related effects, an amelioration of symptoms and disease-related effects, and an increased survival time of the affected host, following administration of the pharmaceutical composition. A prophylactic effect may include a complete or partial avoidance/inhibition or a delay of the pulmonary disease or disorder.

The dose of the Surf-LNP composition may be determined by a medical practitioner in consideration of patient characteristics such as age, weight, gender, symptoms, complications, and the incidence of other diseases. Further, the temporal interval of administration and the number of administrations may be determined in consideration of the dosage form that is used, the half-life of the active ingredient in the lung, and the like.

The exact amount of the composition required may vary from subject to subject, depending on the species, age, weight and general condition of the subject and its mode of administration and the like. Thus, it is not possible to specify an exact amount for every composition. However, an appropriate amount can be determined by one of skill in the art using only routine experimentation given the teachings herein. For example, effective dosages and schedules for administering the compositions may be determined empirically, and making such determinations is within the skill in the art. The dosage ranges for the administration of the compositions are large enough to produce the desired effect in which the symptoms of the disorder are affected. The dosage should not be so large as to cause adverse side effects, such as unwanted cross-reactions, anaphylactic reactions, and the like. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

Toxicity or efficacy of Surf-LNPs can be determined by standard procedures in cell cultures or experimental animals. Data obtained from cell culture assays and laboratory animal studies can be used in formulating a range of dosage for use in humans. The dosage of such components lies, for example, within a range of administered concentrations that include efficacy with little or no toxicity. The dosage may vary within this range depending upon the dosage form employed, the route of administration utilized, and the PK/PD profile of the subject.

Methods for mucosal delivery include those methods known in the art that provide delivery of the composition to mucous membranes. Mucosal delivery methods include intranasal, and oral. In some embodiments, the administration is intranasal. In these embodiments, the Surf-LNP compositions may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages or may be atomized or nebulized for inhalation through the nose or mouth.

Surf-LNP pharmaceutical compositions can optionally be co-administered with one or more additional active agents. Co-administration can include the simultaneous and/or sequential administration of one or more additional active agents and the Surf-LNPs. The one or more additional active agents and the Surf-LNPs can be included in the same or different pharmaceutical formulation. In one aspect, the Surf-LNPs can optionally contain one or more mucus degrading agents. Suitable mucus degrading agents include N-acetylcysteine (NAC), mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4, neltenexine, erdosteine and denufosol. Surf-LNPs can optionally contain one or more anti-infective agents. In certain embodiments, Surf-LNPs contain one or more antibiotics; contain one or more inhaled corticosteroids, such as flunisolide, triamcinolone acetonide, beclomethasone dipropionate, mometasone, budesonide, ciclesonide, or fluticasone propionate; anti-inflammatory molecules and chemotherapeutic agents, and anti-proliferative agents.

The present disclosure further provides immunogenic compositions comprising a Surf-LNP and a nucleic acid encoding for a desired antigen or hapten. In embodiments, the antigen of hapten is derived from derived from a pathogen, an allergen or self-antigen; or derived from a cancer cell.

Such vaccine compositions can be used for protecting a subject against a pathogen of interest. Such pathogens include, for example, influenza virus, poliovirus, rabies virus, hepatitis A and B viruses, measles, mumps, and rubella viruses, Varicella-zoster virus, Yellow fever virus, SARS-CoV-2 virus, Dengue virus, Human papillomavirus); Respiratory syncytial virus), Japanese encephalitis virus, Rotavirus, Meningococcal meningitis). Diphtheria, Tetanus, and Pertussis bacteria, Haemophilus influenzae type b bacteria; Pneumococcal bacteria; Meningococcal bacteria, Shigella, Staphylococcus aureus and non-typhoidal Salmonella. Toxins include those produced by tetanus, diphtheria and Clostridium botulinum bacteria and ricin toxin.

As used herein the term vaccine refers to a composition capable of eliciting in a patient a beneficial active or passive immune response to a specific antigen. While protective immunity may be desired, it is understood that various levels of temporal immune response can be beneficial. Accordingly, use of an effective amount of the nanoparticle for the manufacture of the immunogenic composition against an antigen or hapten, and use of an effective amount of the immunogenic composition for the manufacture of a vaccine for protecting a subject against the antigen or hapten, are provided. In each of the methods of generating an immune response of the present disclosure, the immune response is preferably a protective immune response.

An “immunologically effective amount” of a vaccine formulation is one that is sufficient to induce an immune response to vaccine components in the subject to which the vaccine formulation is administered. A “protective immune response” is one that confers on the subject to which the vaccine formulation is administered protective immunity against the pathogen from which the antigens or haptens of the formulation were obtained. The protective immunity may be partial or complete immunity.

For immunogenic compositions, said compositions may further comprise an immunogenic adjuvant or immunopotentiator. As used herein, the “immunogenic adjuvant” is a substance or composition which can induce and/or enhance an immune response against an antigen. As used the “immunopotentiator” refers to an agent that on inoculation enhances the immune response. Exemplary immunogenic adjuvant can be Freund's complete adjuvant which is a mixture of light mineral oil, Arlacel detergent, and inactivated Mycobacterium tuberculosis bacilli. Exemplary immunopotentiator includes Bacille Calrnette-Guerin (BCG), Corynebacterium Parvum, Brucella abortus extract, glucan, levamisole, tilorone, an enzyme and a non-virulent virus or nucleic acids encoding for immunomodulating molecules.

In another aspect of the embodiment, an article of manufacture (e.g., a kit) containing materials useful for the treatment or prevention of diseases or disorders treatable by administration of the Surf-LNPs, as described above, is provided. The article of manufacture comprises a container and a label or package insert on or associated with the container. Suitable containers include, for example, bottles, vials, syringes, IV solution bags, etc. The containers may be formed from a variety of materials such as glass or plastic. The article of manufacture may comprise a composition contained therein, wherein the composition comprises a Surf-LNP. Kits in certain embodiments may further comprise a package insert indicating that the compositions can be used to treat a particular condition. Alternatively, or additionally, the kit may further comprise a second (or third) container comprising a pharmaceutically acceptable buffer.

All documents, papers and published materials referenced herein, including books, journal articles, manuals, patent applications, published patent applications and patents, are expressly incorporated herein by reference in their entireties.

### EXAMPLE 1

Pulmonary delivery of lipid nanoparticles (LNPs) remains an area of significant interest, given the broad range of genetic disorders that could be addressed through localized administration of therapeutic nucleic acids to the lung. In the present example, the impact of incorporating a native lung component, specifically the clinically used lung surfactant cocktail Poractant alfa, into mRNA-loaded LNPs on their in vitro and in vivo transfection performance was investigated. The resulting lung surfactant-enhanced LNPs (Surf-LNPs) exhibited substantial improvements in particle assembly, yielding an order of magnitude higher particle concentration at equivalent input conditions compared to conventional (Onpattro-like) LNP formulations. In vitro, Surf-LNPs demonstrated several-fold increases in mRNA transfection efficiency and protein expression while maintaining excellent cytocompatibility. These enhancements are attributed, at least in part, to an elevated apparent pKa, which facilitates more rapid and efficient endosomal escape relative to conventional LNPs. In vivo evaluation following intranasal administration further revealed enhanced mCherry expression in the lungs of mice treated with Surf-LNPs compared to conventional LNPs. These findings establish lung surfactant incorporation as a simple yet powerful formulation strategy to improve pulmonary gene delivery using LNPs, with the ability to significantly advance the translation of inhaled nucleic acid therapeutics.

### Materials & Methods

Nanoparticle formulation. LNPs/Surf-LNPs were prepared as follows: an ethanolic solution of Dlin-MC3-DMA (MedChemExpress, NJ; USA), Cholesterol (Sigma Aldrich, USA), DSPC (Avanti Polar lipids, AL, USA) (for LNPs) or Proactant-α (for surf LNPs, calculated based of DPPC content) (Creative Biolabs, NY, USA), DSPE-PEG2000K (Avanti Polar Lipids . . . ) was prepared at molar ratios of 50%, 38.5%, 10%, 1.5%, respectively. Messenger RNA (mEGFP for in-vitro assays, mCherry for in-vivo assays) (Trilink, . . . ) was dispensed in an acetate buffer (100 mM, pH=5). The ethanolic and aqueous phases were mixed by pipetting at an N/P ratio of 6, and an aqueous to organic phase ratio of 3:1 v/v. Immediately after, particle suspension was diluted 10× using Dulbecco's Phosphate Buffer Saline (DPBS, pH=7.4). The diluted suspension was then washed twice with centrifugal filtration using Amikon 100 k and re-diluted to the original volume using DPBS.

DiD labelled particles were prepared similarly except that a 0.3% molar ratio of DiD (Sigma Aldrich) was included in the lipid mixture. That contribution was deduced from the molar contribution of cholesterol in the non-labelled particle variety.

Colloidal properties. Particles were analysed for their size, count, and zeta potential using Nanoparticle Tracking Analysis (NTA) (ZetaView®, Particle Metrix, NC, USA). Briefly, either LNPs or Surf-LNPs were diluted 1000-fold in DPBS, and videos of the particles were acquired at 25 °C at 11 different focal positions within the sample.

Particle stability in simulated lung fluid (Gamble's solution). Surf-LNPs or LNPs prepared as previously described were incubated in Gamble's solution (Pickering Laboratories Inc., CA, USA) at a ratio of 1:10 v/v, at 37 °C with shaking at 70 RPM for 30 minutes. Following this, the incubation solution was diluted 300-fold in DPBS to quench the reaction. Particle count was then assessed using NTA as previously described.

Encapsulation efficiency. LNPs and Surf-LNPs were analyzed for their mRNA encapsulation efficiency using the Ribogreen assay. Either particle was prepared as previously described. The filtrate passing through the Amikon was collected and analyzed for unencapsulated mRNA content using the Ribogreen assay according to the manufacturer's protocol. The amount of free mRNA interacting with Ribogreen dye was assessed spectrophotometrically at λₑₓ=500 nm, λₑₘ=525 nm using Multimode Microplate Reader (Tecan, Männedorf, Switzerland). Then the encapsulation efficiency was calculated according to Equation (1)

$$Equation ( 1 ) = \frac{Total mRNA - Unencapsulated mRNA}{Total mRNA} \times 100$$

Apparent pKa. Apparent pKa was assessed for LNPs or Surf-LNPs using the Lipid Launch Apparent pKa kit (Cayman Chemical, MI, USA). Briefly, Surf-LNP or LNP were prepared as previously described. Samples were washed twice using Amikon 100 k MWCO centrifugal filter (Millipore Sigma, USA) with DPBS at 2150 g for 5 mins. Particles were brought to a final concentration of 1.3 μg mRNA/μL. Surf-LNPs were then diluted 20×, whereas LNPs were used undiluted. In 96-well plates, particle suspensions were mixed with the kit-provided buffers set at an 18:1 v/v ratio with the following pH gradient: 4, 4.5, 5, 5.5, 6, 6.4, 6.8, 7.2, 7.6, 8, 8.5, and 9. Following this, the TNS probe solution was added to the reaction mixture at a 1:20 v/v ratio, and the reaction was allowed to proceed for 20 minutes on an orbital shaker at 60 RPM. The fluorescent signal of the samples was assessed using a plate reader (λₑₓ=320 nm, λₑₘ=450 nm). Average relative fluorescence (ARF) is determined by subtracting the average fluorescence of the pH 9.0 wells from all other wells of a sample. ARF is then plotted against pH, and pKa is calculated at the pH corresponding to 50% maximal fluorescence.

In-vitro Transfection. Adenocarcinoma human alveolar basal epithelial cells (A549) and an immortalized human airway basal cell line (BCi-NS1) were used for the studies. A549 cells were passaged, grown, and maintained in F12K medium supplemented with 10% FBS and 1% Pen/Strep. Bci-NS1 were passaged, grown, and maintained in EX-plus medium supplemented with PneumaCult™-Ex Plus 50×Supplement, 96 μg/L hydrocortisone (Stem Cell Technologies, MA, USA), 1% Pen/Strep, 0.5% Amphotericin (Sigma Aldrich, USA). Cells were consistently kept at 37 °C and 5% CO₂. In either cell line, the transfection procedure was the same. Cells were seeded at a density of 25000 cells/well in 24-well plates. They were allowed to grow to 70-80% confluence over the course of 48 h in their respective growth medium. Cells were then treated with either LNPs, Surf-LNPs, or JetMessenger (Polyplus, NY, USA) with mRNA doses of 1 μg/well in OptiMEM for 24 h. Cells incubated in OptiMEM (Thermo Fisher Scientific, USA) without treatment served as the untreated control. Afterwards, OptiMEM, with or without treatment, is removed, cells are washed twice with DPBS, and are further incubated in their standard growth medium for another 24 h. Eventually, cells were detached with Trypsin-EDTA (Sigma Aldrich, USA) and analyzed for their mEGFP expression using FACS Celesta (BD Biosciences, CA, USA).

Endosomal Escape kinetics. BCis-NS1 were used for endosomal escape kinetic studies. Cells were seeded as previously described for in vitro transfection trials, except that they were seeded on top of microscopic cover slides in the 24-well plates. Cells were then treated with either DiD-labelled LNPs or Surf-LNPs in OptiMEM (1 μg mRNA/well). At 2 h, 6 h, or 24 h, treatments were removed, and cells were stained with 80 nM Lysotracker Yellow HCK-123 (Thermo Fisher Scientific, USA) in the previously described BCi-NS1 growth medium for 1 h at 37 °C, 5% CO2. Following this, the respective coverslides were retrieved, mounted on microscopic slides using Vectashield (Vector Labs, CA, USA), and visualized using Confocal Laser Scanning Microscopy (Zeiss LSM 780, Zeiss, USA). The acquired images were analysed for DiD (particle) and Lysotracker (endolysosome) colocalization over time using the Colocalization Threshold plugin on ImageJ.

Intranasal delivery of LNPs. Twelve 8-week-old female BALB-c mice were intranasally inoculated with either DPBS (n=2), LNPs (n=5), or Surf-LNPs (n=5). Both LNP and Surf-LNP mice received an mCherry dose of 2.5 μg mRNA per animal in a total volume of 20 μL (10 μL/nostril). Doses were administered using a 10 μL pipette to the animal under anaesthesia. After 24 h, live animals were visualized using IVIS ( ) After either 6 h or 24 h, animals were immediately sacrificed. Afterwards, lungs (at 24 h) and trachea (at 6 h) were harvested.

For quantitative assessment of mCherry expression, lungs were homogenized in T-PER lysis buffer (Thermo Fisher Scientific) supplemented with protease inhibitor cocktail (Sigma Aldrich). Briefly, lungs were mixed with the buffer at 5% w/v and diced using a Pestle Motor Mixer. Tissue homogenates were then centrifuged at 10000 g for 5 mins. Supernatant was spectrophotometrically assessed for mCherry fluorescence at λₑₓ=587 nm, λₑₘ=610 nm using Multimode Microplate Reader (Tecan, Männedorf, Switzerland).

Flow cytometric analysis of mCherry expression in immune vs epithelial cell populations was performed. Briefly, Lung tissue was finely diced, then incubated in a solution of collagenase IV (0.5 mg/mL) (Sigma-Aldrich, USA) and DNase I (10 μg/mL) (Sigma-Aldrich) in RPMI at 37 °C, 5% CO2, shaking at 50 RPM for 45 minutes. Tissue digest was then passed through a 70 μm cell strainer (Millipore Sigma, USA). Cells were then collected by centrifugation at 300 g for 10 minutes. The pellet was washed with PBS, then treated with RBC lysis buffer for 3 minutes on ice. The reaction was halted by 4×dilution in cold PBS, followed by washing.

Cells were stained for either CD45 or EpCAM as follows. First, cells were incubated with CD16/CD32 Monoclonal antibody for 10 minutes on ice to perform Fc blocking. Cells were then washed with PBS and incubated with either FITC-labelled anti-CD45 or anti-EpCAM[SN1] [SN2] (Invitrogen, Thermo Fisher Scientific, USA) for 45 minutes on ice. Cells were washed and permeabilized for intracellular staining using Cytoperm/Wash for 15 minutes. Cells were then incubated with Alexa Fluor 647 labeled anti-mCherry monoclonal antibody (Invitrogen, Thermo Fisher Scientific, USA) for 30 minutes on ice. Cells were then analyzed using FACS Celesta (BD Biosciences, CA, USA). For confocal imaging, lungs or tracheas from each treatment group were mounted in OCT, sectioned on a cryomicrotome, and mounted on microscopic slides. The mounted tissue was fixed with 4% PFA, washed with PBS, permeabilized with 0.5% Triton X-100 (v/v) in PBS, and then stained with 300 nM DAPI (Sigma Aldrich, USA) in PBS. The tissues were then imaged with CLSM to visualize mCherry expression. % mCherry-expressing cells, relative to the total number of cells per image, were calculated from CLSM images using ImageJ, with the untreated control used to set the threshold for mCherry expression.

### Results

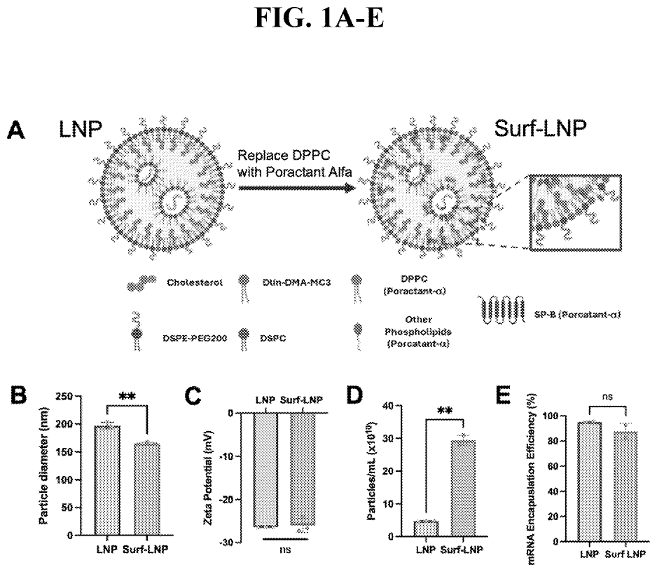

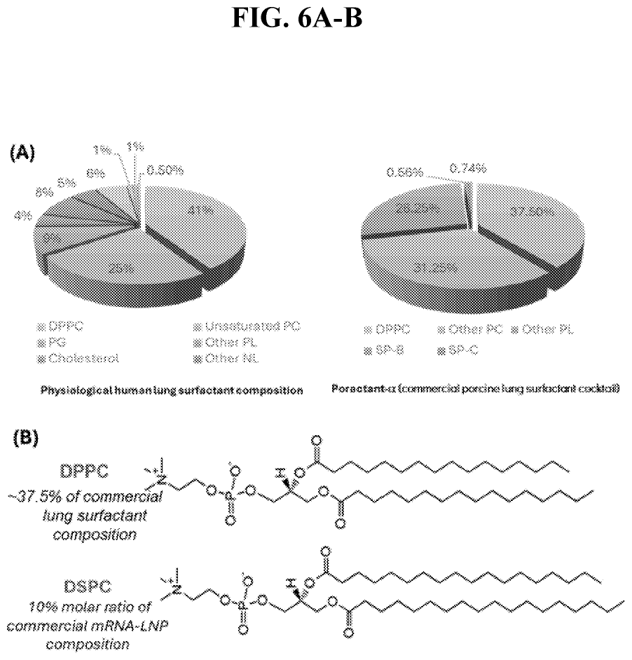

LNPs were engineered where distearoylphosphatidylcholine (DSPC), the standard helper lipid in many commercially available LNPs, was replaced with Poractant-α, a porcine lung surfactant cocktail (Table 1; FIG. 1A). These Poractant-α containing LNPs are referred to as “Surf-LNPs”. Poractant-α is a natural lung surfactant used to treat respiratory distress syndrome (RDS) in premature infants²⁸. Its composition features a simplified yet physiologically relevant assortment of phospholipids and lipoproteins (FIG. 6A)²⁹. Dipalmitoyl phosphatidylcholine (DPPC) is the main phospholipid component in poractant-α (37.5% molar ratio) and is very similar to DSPC. Both DSPC and DPPC are saturated phospholipids with the same choline headgroup, with the only structural difference being a shorter (C₁₆) hydrocarbon tail in the case of DPPC compared to DSPC (C₁₈) (FIG. 6B).

TABLE 1
Composition of LNPs and Surf-LNPs, represented as %
molar ratios of the enlisted lipids and the universal
N/P ratio used to prepare both particle types.

| | LNP | Surf-LNP |
|---|---|---|
| Dlin-MC3-DMA | 50% | 50% |
| Cholesterol | 38.5% | 38.5% |
| DSPE-PEG2000K | 1.5% | 1.5% |
| DSPC | 10% | — |
| Poractant-α | — | 10% (calculated as DPPC) |
| N/P ratio | 6 | 6 |

Merckx et al.¹ used either a lipid cocktail or poractant-α to coat an siRNA-dextran nanogel. The lipid cocktail used was DPPC and PG, the main phospholipid components of porcine surfactant alpha, making SP-B and SP-C the only unique components when compared to poractant-α. Yet they showed that, while the lipid cocktail dramatically reduced particle uptake and gene silencing activity compared to non-coated particles in vitro, poractant-α reduced uptake to a slightly lesser extent and maintained gene silencing efficiency that was almost equivalent to that of the non-coated particles. Similarly, in vitro results were also observed when gelatin-DNA nanogels were coated with an LNP lipid mixture; the coated particles showed negligible transfection efficiency³. The gene silencing capacity in vivo for this nanogel system was most pronounced for SP-B-containing lipid cocktails, which were closer in composition to the full poractant-α cocktail, whereas transfection by non-coated nanogels was virtually undetectable. Nanogels coated with the lipid cocktail exhibited only a modest gene-silencing effect compared to their SP-B-containing counterparts. It was thus demonstrated that SP-B was an efficient RNA delivery enhancer when incorporated into a suitable phospholipid mixture.

Surfactant Protein B (SP-B) is a strongly hydrophobic protein, yet it also contains a substantial fraction of basic amino acids, lending it a net positive charge. Its mature form is a homodimer featuring two chains, each 79 amino acids long⁴. Along with the lipid components of lung surfactant, SP-B plays a crucial role in stabilizing the surface tension at the pulmonary air-liquid interface, preventing lung collapse during respiration^{5,6}. Each monomer of the homodimer is comprised of 4-5 amphipathic α-helices, stabilized by three intramolecular disulfide bonds. The dimer is then formed by one intermolecular disulfide bond between the two monomers⁷. This tertiary structure is homologous to saposin-like proteins (SALIPs)⁸. The characteristic SALIP fold comprises 4-5 α-helical hairpin units that facilitate its interaction with lipid membranes⁹. Where the hydrophobic axes of the helices featuring mainly the hydrophobic amino acids are oriented parallel to the lipid tail's interface, and the protein's positive charges, endowed by the basic amino acids, are thought to interact with the anionic heads of phospholipids^{4,10,11}. It was believed that this could represent the primary mode of SP-B incorporation into LNPs (FIG. 1A).

LNP and Surf-LNPs were prepared at an N/P ratio of 6 and an aqueous-to-organic phase ratio of 1:1, and analysed for their size, particle concentration, and zeta potential using Nanoparticle Tracking Analysis (NTA). Surf-LNPs demonstrated a slight yet statistically significant reduction in particle diameter compared to conventional LNPs (FIG. 1B). Both systems showed comparable zeta potentials (~−25 mV) (FIG. 1C). However, even at the same effective mRNA/lipid concentration, a roughly order of magnitude increase in particle count of Surf-LNPs compared to conventional LNPs was observed (FIG. 1D). The significant increase in particle count could be attributed to SP-B's structural properties. The slight positive charge on SP-B (+5-+8)¹⁰ could enable closer interaction with the mRNA cargo and faster, more efficient compaction than fully neutral helper lipid components. This could result in a more efficient, compact assembly of Surf-LNP particles, potentially yielding a higher particle count per unit mass of lipid components than conventional LNPs lacking SP-B. Both systems showed comparable and acceptable mRNA encapsulation efficiencies (FIG. 1E).

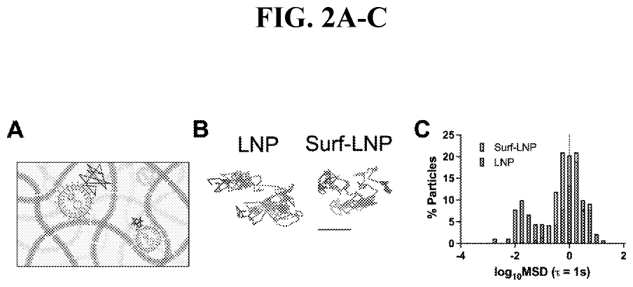

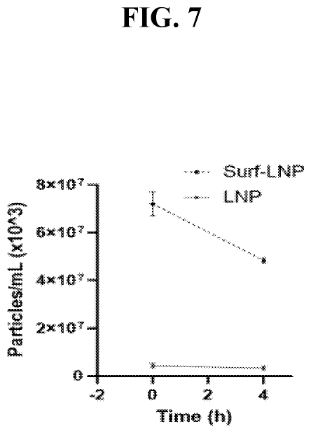

To assess particles' stability in a physiologically relevant medium, Surf-LNPs or LNPs were prepared and incubated in simulated lung fluid (Gamble's solution). While Surf-LNP remained at an order of magnitude higher total particle concentration, Surf-LNPs showed apparently lower colloidal stability with a 32.7±3.3% reduction in particle count, opposed to 23.1±10.4% in the case of LNPs after 4 h of incubation (FIG. 7). To next consider the barrier function of mucus towards pulmonary LNP delivery, NP diffusion in secreted airway mucus was evaluated using fluorescence video microscopy and multiple-particle tracking, as described in previous work (FIG. 2A)^{12,13}. Airway mucus was obtained from air-liquid interface cultures of differentiated BCi-NS1 lung epithelial cells using previously established protocols. DiD-labeled LNPs or Surf-LNPs were added to mucus and imaged using confocal microscopy to evaluate their mobility in the mucus barrier. Representative trajectories show both LNP types were highly diffusive, presumably due to the DSPE-PEG shielding these particles from mucoadhesion and trapping in the mucus gel. From these trajectories, the mean squared displacement (MSD) for each particle was determined as MSD(τ)=((x²+y²)). Based on prior work illustrating how NP diffusion through mucus, as assessed by MPT, translates to in vivo biodistribution in the airways¹⁴⁻¹⁶, particles highly mobile and likely to rapidly penetrate the mucus barrier were considered if they achieved an MSD (τ=1 s) that is equal to or exceeds 1 μm². While Surf-LNPs appeared to perform more consistently across measurements than LNP (FIG. 2C), both systems showed comparable mobility in mucus with the majority of individual particles (>50%) able to rapidly diffuse through mucus.

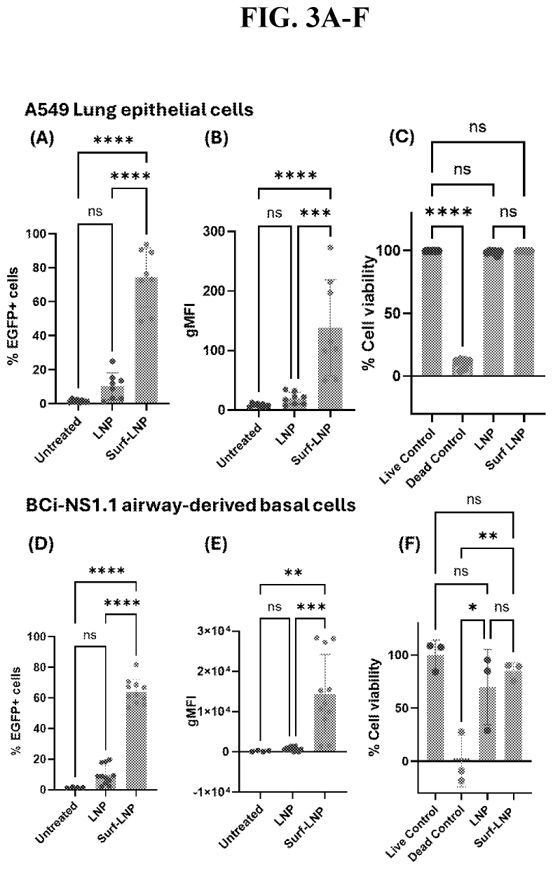

To compare the mRNA delivery efficiency of LNP and Surf-LNP, two cell lines were used: adenocarcinoma human alveolar epithelial cells (A549) and human airway basal cells (BCi-NS1). Each cell type was treated with either LNPs or Surf-LNPs at an mRNA dose of 1 μg/well for 24 h. In A549 cells, Surf-LNPs showed ~8.5-fold increases in transfection efficiency compared to LNPs (FIG. 3A), as well as a ~7-fold increase in protein expression (FIG. 3B). Both Surf-LNPs and LNPs demonstrated excellent cytocompatibility, with cell viability comparable to that of non-treated cells (FIG. 3C). Similar trends were observed in BCi.NS1, where Surf-LNPs showed ~6.5-fold increase in transfection efficiency (FIG. 3D), and 4 orders of magnitude increase in level of protein expression (FIG. 3E) compared to LNPs. Both particles demonstrated acceptable viability, with Surf-LNP showing slightly higher, though statistically non-significant cytocompatibility (FIG. 3F). The significant improvement in the ease of particle assembly, and hence a much higher particle count for Surf-LNPs at similar lipid and mRNA concentrations to conventional LNPs, may have contributed to the system's enhanced in vitro transfection performance. Yet the previously reported endosomal escape-enhancing capabilities of poractant-α and its lipoprotein component, SP-B, may also have played a crucial role. An attempt to assess this is disclosed below.

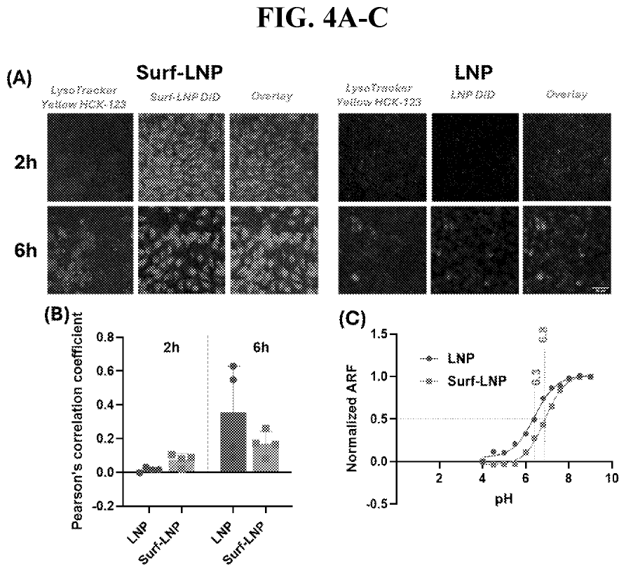

The assessment of endosomal escape kinetics demonstrated higher as well as earlier uptake of DiD-labeled Surf-LNPs compared to plain LNPs (FIG. 4A). As early as 2 h, prominent uptake of Surf-LNPs was observed; particles appeared uniformly distributed in the cytosol. However, the endosomal compartment proved to be tricky to stain at that time point. This could be due to early and efficient endosomal escape at this stage, leaving very few endosomal compartments intact. In contrast, DiD-labeled LNPs showed lower uptake at localized spots, and staining of endosomal compartments was feasible at 2 h, presumably indicating slower, less efficient uptake and endosomal escape than with Surf-LNPs. At 6 h, LNPs demonstrated a steady increase in uptake compared to their 2 h point, yet they also demonstrated higher endosomal entrapment than Surf-LNPs (FIG. 4B).

LNPs have also demonstrated a lower pKa (6.3) than Surf-LNPs (6.8) (FIG. 4C), which is also consistent with the earlier endosomal escape observed for Surf-LNPs. It was thought that the higher apparent pKa of Surf-LNPs compared to LNPs could be attributed to the overall cationic charge of SP-B and SP-C in Proactant-α. Both lipoproteins could be stacking high up the lipid tails of anionic lipids and close to their anionic heads, involving these heads in subtle electrostatic interactions, which could render the ionizable lipids more susceptible to protonation at a higher pH than a lipid film where anionic lipids are more available to compete for the proton with the neighboring ionizable lipid. The earlier endosomal escape of Surf-LNP could thus be attributed to a range of mechanisms, in which the higher apparent pKa of Surf-LNP enables it to fuse with the endosomal lipid bilayer, thereby converting it to a hexagonal state more rapidly^{17,18} Simultaneously, as SP-B leaks from the particle, it can form channels in the endosomal membrane, further accelerating endosomal escape. Once the pH drops sufficiently in late endosomes, SP-B begins to attack the anionic components of the limiting membrane and ILVs, and escapes via either direct or back fusion.

SP-B has been identified as an enhancer of gene delivery by acting as a potent endosomal escape enhancer. As an endosome matures, late endosomes become richer in anionic endogenous lipids such as bis(monoacylglycero)phosphate, phosphatidylserine, and phosphatidylinositol. Such anionic lipids are present both in the outer membrane of the late endosome and its ILVs present in late endosomes. Considering this, a dual mechanism was proposed by which SP-B-containing particles escape the endosome, both by virtue of SP-B's cationic charge interacting with and disrupting the anionic lipids of either the outer membrane allowing the escape of the RNA cargo directly into the cytosol “direct fusion” or through fusion with the ILVs membranes, followed by “back fusion” with the limiting membrane and into the cytosol². Biophysical evaluation has further demonstrated the essential role of SP-B in promoting the absorption of lung surfactant lipid components to the alveolar air-liquid interface as multilamellar films^{19,20} This function is facilitated by the fusogenic and surface-active properties of SP-B, which enable it to pack lipid films more closely together. Recent studies have also demonstrated that SP-B dimers tend to assemble into ring-like nanosized multimers through lateral migration in lipid films, especially in non-saturated lipids. Additionally, such rings can stack as channels that connect these lipid films in multilayers²¹⁻²³. This mechanism could practically allow SP-B, once liberated from the Surf-LNP, to ‘punch nano-pores’ in the endosomal membrane that could facilitate mRNA escape. To this end, these functions can be evaluated in LNP formulations with substitution of helper lipids with DSPC and inclusion of SP-B to compare to the Surf-LNP formulation.

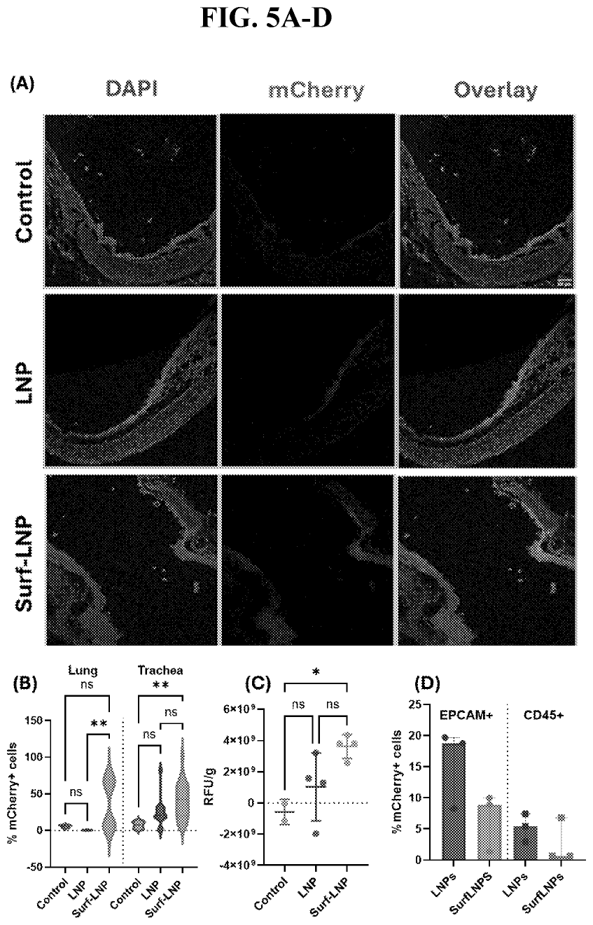

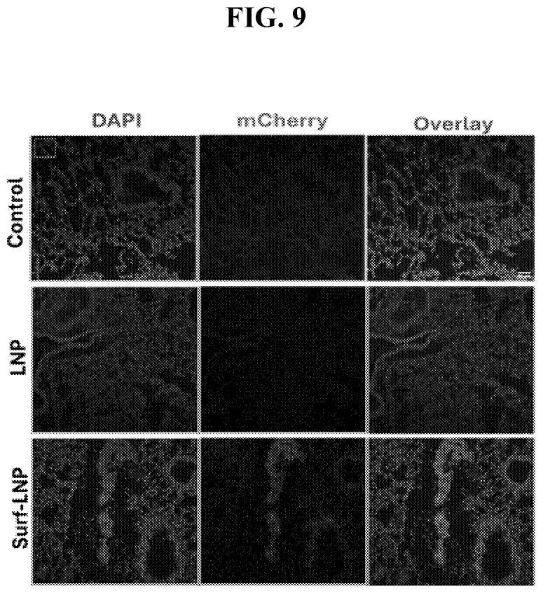

To further evaluate pulmonary mRNA delivery in vivo, 8-week-old female BALB-c mice were intranasally inoculated with either DPBS (n=2), LNPs (n=5), or Surf-LNPs (n=5). Both LNP and Surf-LNP mice received an mCherry dose of 2.5 μg mRNA. It is noted this dose is lower than many previous studies on intranasal LNP delivery but was selected based on previous reports showing substantial inflammation at doses >0.5 μg mRNA following intranasal delivery²⁴. After either 6 h or 24 h, the animals were sacrificed immediately, and lung tissue was harvested for subsequent analysis. Based upon confocal imaging of lung tissue section, greater mCherry expression was observed in the tracheal lining following Surf-LNP administration as opposed to LNPs (FIG. 5A-B). The same trend was also observed in the lung parenchyma (FIG. 9), which demonstrated significantly higher expression of Mcherry compared to LNP (FIG. 5B), though most expression was observed around bronchioles.

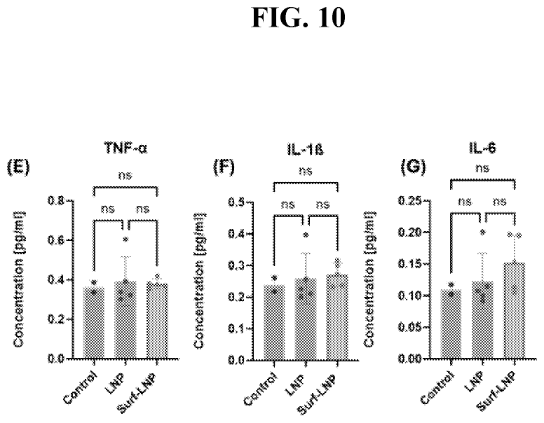

Tissue homogenates were also prepared for bulk assessment of mCherry expression and flow cytometry was performed to assess reporter expression in immune (CD45+) versus epithelial (EPCAM+) cell populations. Whole lung homogenates from tissues collected 24 h post-treatment showed a significant increase in mCherry expression in Surf-LNP treated groups compared to untreated controls (FIG. 5B). LNP showed a more variable expression between replicates and no significant difference in the level of mCherry expression with either PBS or Surf-LNP-treated groups. Interestingly, flow cytometric analysis of mCherry expression in EPCAM+ vs CD45+ cells in a single cell suspension of the lung tissue showed that LNP transfected both EPCAM+ and CD45+ cells, yet Surf-LNP appeared to predominantly transfect EPCAM+ cells (FIG. 6C). More data is still required to fully elucidate this finding, yet several accounts in the literature have demonstrated the propensity of smaller nanoparticles <100-200 nm to be taken up by endocytosis by non-immunogenic cell populations (such as epithelial cells), as opposed to larger (>200 nm) particles more commonly taken up phagocytosis by CD45+ macrophages or other antigen presenting cells²⁵⁻²⁷. Inflammatory cytokine response assessment in the supernatant of lung homogenates after 6 h of particle treatment demonstrated that neither LNPs nor Surf-LNPs induced a significant increase in inflammatory cytokines (TNF-α, IL-1β, IL-6) compared to the PBS-inoculated group (FIG. 10).

In conclusion, it is demonstrated herein that replacing the standard helper lipid (DSPC) of commercial LNPs with a biomimetic helper lipid cocktail of commercially available lung surfactant (poractant-α) resulted in a logarithmic increase in particle count due to improved particle assembly. This improvement could be attributed to the unique saposin-like structure of SP-B, which may have promoted a more compact and efficient packing of the surfactant-doped Surf-LNP compared to conventional LNP. Furthermore, the resulting Surf-LNP showed earlier and higher endosomal escape, potentially due to the superior fusogenic properties of SP-B, as well as the elevated apparent pKa of Surf-LNPs compared to LNPs. Due to enhancement in endosomal escape, Surf-LNPs significantly outperformed conventional LNPs in both transfection efficiency and protein expression levels. The findings highlight that incorporating lung-derived helper and structural LNP components, which actively facilitate endosomal escape, could enhance LNP performance. A ~2-fold improvement in mRNA expression in vivo following intranasal delivery of Surf-LNPs as compared to LNPs and a transfection profile that notably favoured epithelial over immune cells was observed.

As such, Surf-LNPs can prove useful for protein replacement therapy applications which would benefit from targeting epithelial cell subsets. Given that this approach could be used in a wide variety of newly emerging LNP formulations with greater potency, one may adapt this formulation approach in combination with other ionizable lipid platforms with the aim of further improving pulmonary mRNA therapeutics for chronic lung diseases.

### REFERENCES

(1) Merckx, P.; Backer, L. D.; Hoecke, L. V.; Guagliardo, R.; Echaide, M.; Baatsen, P.; Olmeda, B.; Saelens, X.; Pérez-Gil, J.; Smedt, S. C. D.; Raemdonck, K. Surfactant Protein B (SP-B) Enhances the Cellular siRNA Delivery of Proteolipid Coated Nanogels for Inhalation Therapy. Acta Biomater. 2018, 78, 236-246.
(2) Guagliardo, R.; Herman, L.; Penders, J.; Zamborlin, A.; Keersmaecker, H. D.; Vyver, T. V. de; Verstraeten, S.; Merckx, P.; Mingeot-Leclercq, M.-P.; Echaide, M.; Pérez-Gil, J.; Stevens, M. M.; Smedt, S. C. D.; Raemdonck, K. Surfactant Protein B Promotes Cytosolic SiRNA Delivery by Adopting a Virus-like Mechanism of Action. ACS Nano 2021, 15 (5), 8095-8109.
(3) Nasr, S. S.; Paul, P.; Loretz, B.; Lehr, C.-M. Realizing Time-Staggered Expression of Nucleic Acid-Encoded Proteins by Co-Delivery of Messenger RNA and Plasmid DNA on a Single Nanocarrier. Drug Deliv. Transl. Res. 2024, 14 (12), 3339-3353.
(4) Veldhuizen, E. J. A.; Haagsman, H. P. Role of Pulmonary Surfactant Components in Surface Film Formation and Dynamics. Biochim. Biophys. Acta (BBA)—Biomembr. 2000, 1467 (2), 255-270.
(5) Guagliardo, R.; Pérez-Gil, J.; Smedt, S. D.; Raemdonck, K. Pulmonary Surfactant and Drug Delivery: Focusing on the Role of Surfactant Proteins. J. Control. Release 2018, 291, 116-126.
(6) Pioselli, B.; Salomone, F.; Mazzola, G.; Amidani, D.; Sgarbi, E.; Amadei, F.; Murgia, X.; Catinella, S.; Villetti, G.; Luca, D. D.; Carnielli, V.; Civelli, M. Pulmonary Surfactant: A Unique Biomaterial with Life-Saving Therapeutic Applications. Curr. Med. Chem. 2022, 29 (3), 526-590.
(7) Liu, J. Y.; Sayes, C. M. Lung Surfactant as a Biophysical Assay for Inhalation Toxicology. Curr. Res. Toxicol. 2022, 4, 100101.
(8) Waring, A. J.; Whitelegge, J. P.; Sharma, S. K.; Gordon, L. M.; Walther, F. J. Emulation of the Structure of the Saposin Protein Fold by a Lung Surfactant Peptide Construct of Surfactant Protein B. PLOS ONE 2022, 17 (11), e0276787.
(9) Morrow, M. R.; Abu-Libdeh, N.; Stewart, J.; Keough, K. M. W. Interaction of Pulmonary Surfactant Protein SP-A with DPPC/Egg-PG Bilayers. Biophys. J. 2003, 85 (4), 2397-2405.
(10) Ding, J.; Takamoto, D. Y.; Nahmen, A. von; Lipp, M. M.; Lee, K. Y. C.; Waring, A. J.; Zasadzinski, J. A. Effects of Lung Surfactant Proteins, SP-B and SP-C, and Palmitic Acid on Monolayer Stability. Biophys. J. 2001, 80 (5), 2262-2272.
(11) Seifert, M.; Breitenstein, D.; Klenz, U.; Meyer, M. C.; Galla, H.-J. Solubility versus Electrostatics: What Determines Lipid/Protein Interaction in Lung Surfactant. Biophys. J. 2007, 93 (4), 1192-1203.
(12) Kaler, L.; Iverson, E.; Bader, S.; Song, D.; Scull, M. A.; Duncan, G. A. Influenza A Virus Diffusion through Mucus Gel Networks. Commun Biology 2022, 5 (1), 249.
(13) Song, D.; Iverson, E.; Kaler, L.; Bader, S.; Scull, M. A.; Duncan, G. A. Modeling Airway Dysfunction in Asthma Using Synthetic Mucus Biomaterials. ACS Biomater. Sci. Eng. 2021, 7 (6), 2723-2733.
(14) Chen, D.; Liu, J.; Wu, J.; Suk, J. S. Enhancing Nanoparticle Penetration through Airway Mucus to Improve Drug Delivery Efficacy in the Lung. Expert Opin. Drug Deliv. 2021, 18 (5), 595-606.
(15) Schneider, C. S.; Xu, Q.; Boylan, N. J.; Chisholm, J.; Tang, B. C.; Schuster, B. S.; Henning, A.; Ensign, L. M.; Lee, E.; Adstamongkonkul, P.; Simons, B. W.; Wang, S.-Y. S.; Gong, X.; Yu, T.; Boyle, M. P.; Suk, J. S.; Hanes, J. Nanoparticles That Do Not Adhere to Mucus Provide Uniform and Long-Lasting Drug Delivery to Airways Following Inhalation. Sci. Adv. 2017, 3 (4), e1601556.
(16) Lock, J. Y.; Carlson, T.; Carrier, R. L. Mucus Models to Evaluate the Diffusion of Drugs and Particles. Adv. drug Deliv. Rev. 2018, 124, 34-49.
(17) Patel, P.; Ibrahim, N. M.; Cheng, K. The Importance of Apparent pKa in the Development of Nanoparticles Encapsulating siRNA and mRNA. Trends Pharmacol. Sci. 2021, 42 (6), 448-460.
(18) Lu, Z.-R.; Sun, D. Mechanism of pH-Sensitive Amphiphilic Endosomal Escape of Ionizable Lipid Nanoparticles for Cytosolic Nucleic Acid Delivery. Pharm. Res. 2025, 42 (7), 1065-1077.
(19) Frey, S. L.; Pocivavsek, L.; Waring, A. J.; Walther, F. J.; Hernandez-Juviel, J. M.; Ruchala, P.; Lee, K. Y. C. Functional Importance of the NH2-Terminal Insertion Sequence of Lung Surfactant Protein B. Am. J. Physiol.—Lung Cell. Mol. Physiol. 2010, 298 (3), L335-L347.
(20) Keating, E.; Zuo, Y. Y.; Tadayyon, S. M.; Petersen, N. O.; Possmayer, F.; Veldhuizen, R. A. W. A Modified Squeeze-out Mechanism for Generating High Surface Pressures with Pulmonary Surfactant. Biochim. Biophys. Acta (BBA)—Biomembr. 2012, 1818 (5), 1225-1234.
(21) Parra, E.; Alcaraz, A.; Cruz, A.; Aguilella, V. M.; Pérez-Gil, J. Hydrophobic Pulmonary Surfactant Proteins SP-B and SP-C Induce Pore Formation in Planar Lipid Membranes: Evidence for Proteolipid Pores. Biophys. J. 2013, 104 (1), 146-155.
(22) Martínez-Calle, M.; Parra-Ortiz, E.; Cruz, A.; Olmeda, B.; Pérez-Gil, J. Towards the Molecular Mechanism of Pulmonary Surfactant Protein SP-B: At the Crossroad of Membrane Permeability and Interfacial Lipid Transfer. J. Mol. Biol. 2021, 433 (3), 166749.
(23) Parra, E.; Moleiro, L. H.; López-Montero, I.; Cruz, A.; Monroy, F.; Pérez-Gil, J. A Combined Action of Pulmonary Surfactant Proteins SP-B and SP-C Modulates Permeability and Dynamics of Phospholipid Membranes. Biochem. J. 2011, 438 (3), 555-564.
(24) Ndeupen, S.; Qin, Z.; Jacobsen, S.; Bouteau, A.; Estanbouli, H.; Igyártó, B. Z. The mRNA-LNP Platform's Lipid Nanoparticle Component Used in Preclinical Vaccine Studies Is Highly Inflammatory. Iscience 2021, 24 (12), 103479.
(25) Tennikova, T.; Urtti, A. Modified Cells as Potential Ocular Drug Delivery Systems. Drug Discov. Today 2019, 24 (8), 1621-1626.
(26) Yu, W.; Liu, R.; Zhou, Y.; Gao, H. Size-Tunable Strategies for a Tumor Targeted Drug Delivery System. ACS Cent. Sci. 2020, 6 (2), 100-116.
(27) Mok, Z. H. The Effect of Particle Size on Drug Bioavailability in Various Parts of the Body. Pharm. Sci. Adv. 2023, 2, 100031.
(28) Guo X et al., In Vitro Characterization and In Vivo Comparison of the Pulmonary Outcomes of Porfactant Alfa and Calsurf in Ventilated Preterm Rabbits PLos ONE 2020 (15 (3). e0230229.
(29) Shelley, S. A. et al., Lung Surfactant Phospholipids in Different Animal Species. Lipids 1984, 19 (11) 857-862.

What is claimed:

1. A lipid nanoparticle comprising one or more lipid component and one or more lung surfactant (Surf-LNP).

2. The Surf-LNP of claim 1, further comprising one or more of the following components: cholesterol and a PEGylated lipid.

3. The Surf-LNP of claim 1, further comprising a nucleic acid molecule.

4. The Surf-LNP of claim 3, wherein the nucleic acid encodes for a therapeutic protein;
a prophylactic protein, anti-inflammatory protein; or an antigen or hapten.

5. The Surf-LNP of claim 4, wherein the nucleic acid encodes an antigen or hapten for use in stimulation of an immune response.

6. The Surf-LNP of claim 3, wherein the nucleic acid encodes for a wild type protein.

7. The Surf-LNP of claim 3, wherein the nucleic acid is a sgRNA for use in a CRISPR/CAS9 system.

8. The Surf-LNP of claim 6, wherein the sgRNA targets the cystic fibrosis transmembrane conductance regulator (CTFR) gene for correction of a genetic defect in said gene.

9. The Surf-LNP of claim 3, wherein the nucleic acid is an inhibitory nucleic acid.

10. The Surf-RNA of claim 9, wherein the inhibitory nucleic acid is selected from the group consisting of an antisense, miRNA, siRNA or shRNA.

11. The Surf-RNA of claim 10 wherein the inhibitory nucleic acid targets a mucin SPDEF transcription factor and/or alarmin gene product.

12. A pharmaceutical composition, comprising the Surf-LNP of claim 1 and a pharmaceutically acceptable carrier or excipient.

13. An immunogenic composition comprising the Surf-LNP of claim 5.

14. The immunogenic composition of claim 13, wherein said composition is used as a vaccine.

15. The immunogenic composition of claim 14, further comprising an adjuvant.

16. A method for treatment or preventing a disease or condition in a subject in need comprising administration of the Surf-LNP of claim 1.

17. The method of claim 16, wherein the disease or condition is pulmonary disease or condition.

18. The method of claim 17, wherein the pulmonary disease is cystic fibrosis.

19. The method of claim 16, wherein the pulmonary disease is asthma.

20. The method of claim 18, wherein the Surf-LNP is a component of a CRISPR/CAS9 system administered for correction of a CFTR gene defect.
